(19) United States
(12) Patent Application Publication
(10) Pub. No.: US 2008/0006282 A1
(43) Pub. Date: Jan. 10, 2008

(54) MEDICAL IMAGING EXCHANGE NETWORK

(76) Inventors: Predrag Sukovic, Birmingham, MI (US); David Phillipe Sarment, Ann Arbor, MI (US); Neal Clinthorne, Ann Arbor, MI (US); Miodrag Rakic, Redondo Beach, CA (US)

Correspondence Address: CARLSON, GASKEY & OLDS, P.C., 400 WEST MAPLE ROAD, SUITE 350, BIRMINGHAM, MI, 48009, US

(21) Appl. No.: 11/744,293

(22) Filed: May 4, 2007

Related U.S. Application Data
(60) Provisional application No. 60/797,565, filed on May 4, 2006.

(51) Int. Cl. A61B 5/05

(52) U.S. Cl. 128/922; 600/425

(57) ABSTRACT

A surgical imaging system includes a CT scanner and an image exchange system. A computer interfaces with an image exchange server over the Internet or over another wide area network. The image exchange server provides an offsite backup of images, provides a complete project management for medical procedures involving the images, and provides software available for trial and purchase for use on the surgical imaging system or other computers.

This application claims priority to United States Provisional Application No. 60/797,565 filed May 4, 2006.

### BACKGROUND OF THE INVENTION

The present invention relates generally to a network for managing medical images and more particularly to a system and method for remotely storing, accessing, processing and distributing medical images.

Medical imaging has widely evolved in the recent years, offering sophisticated tests such as Computer Tomography (CT), Magnetic Resonance Imaging (MRI), ultrasounds, or the like. Manufacturers such as Xoran Technologies, Inc. produce a wide variety of such medical imaging devices including compact CT scanners ideally suited for in-office or on-site imaging of numerous areas of the human body, including but not limited to the temporal bones, the skull base, sinuses, and jaw/teeth.

Typically, users of compact imaging equipment of this type perform a CT scan and study, view and store the resulting image at the location where the CT scan was performed. The CT image may also need to be viewed by other doctors or specialists for a second opinion or by other specialists such as radiologists, dental implant manufactures and drill implant manufacturers. The current procedure for providing these necessary parties with the patient's CT image typically involves copying the image to a compact disk or other similar storage medium and shipping the image to the appropriate party.

Disadvantageously, this process may be time consuming and expensive. For example, it may take days for a doctor or specialist to receive the scanned image, review and analyze the image, and forward the image to the next doctor or physician required to review the CT image. Also, “emailing” CT images is currently not feasible due to the extremely large size of the image files. This further makes the current process of distributing the scanned images impractical.

Accordingly, it is desirable to provide a system of storing, accessing and distributing medical images for users of medical imaging equipment that is simple and cost-effective to operate and that provides for remote viewing of medical images in a secured and controllable manner.

### SUMMARY OF THE INVENTION

A surgical imaging system includes a CT scanner and an image exchange system. A computer interfaces with an image exchange server over the Internet or over another wide area network. Other users and computers can access the image exchange server. The image exchange server provides an offsite backup of images, provides a complete project management for medical procedures involving the images and provides software available for trial and purchase for use on the surgical imaging system or other computers.

Copies of CT images stored on the computer are periodically and automatically copied by software on the computer to the image exchange server and associated with a physician's account. The CT images on the image exchange server are accessible only by the physician's account and those accounts authorized by the physician. The physician handling the patient grants the authority to remotely view selected CT images through the image exchange server to the patient, other physicians, or specialists.

The image exchange server issues a notification to the outside contractors that access to the selected CT image(s) has been granted and specific work is requested of them. The outside contractor may either accept or reject the request, and the physician is notified of the acceptance or rejection.

Upon acceptance of the request to perform services, the image exchange server grants the outside contractor access to the CT images of the patient. Based on the CT images, the outside contractors may then add comments or markings to the CT images, design or locate implants within the CT images, or perform any other required services on or with the CT images. If the outside contractor rejects the request, the image exchange server notifies the handling physician, and another outside contractor is chosen to perform the required services.

The image exchange server monitors the progress of the outside contractors as the requested services are performed. The image exchange server notifies the handling physician of the progress of the outside contractor. The handling physician reviews the services performed by the outside contractors and makes any necessary preparations for performance of the required medical procedure.

The image exchange server can also coordinate payments and provide software that can be downloaded to other computers.

### BRIEF DESCRIPTION OF THE DRAWINGS

The various features and advantages of the present invention will become apparent to those skilled in the art from the following detailed description of the currently preferred embodiment. The drawings that accompany the detailed description can be briefly described as follows:

FIG. 1 schematically illustrates a first embodiment CT scanner;

FIG. 2 illustrates the CT scanner of FIG. 1 with a part of a patient received in the CT scanner;

FIG. 3 illustrates a second embodiment of the CT scanner;

FIG. 4 illustrates a perspective view of a CT scanning system including the CT scanner, a computer and an image exchange system;

FIG. 5 is a block diagram illustrating a method of storing, accessing and distributing CT images;

FIG. 6 illustrates a screen capture of a login page used to access an image exchange network;

FIG. 7 illustrates a screen capture of a patient search page for searching for a patient on the image exchange network;

FIG. 8 illustrates a screen capture of a page that displays records of a patient; and

FIG. 9 illustrates a screen capture of a page used to provide outside contractor authorization.

### DETAILED DESCRIPTION OF PREFERRED EMBODIMENTS

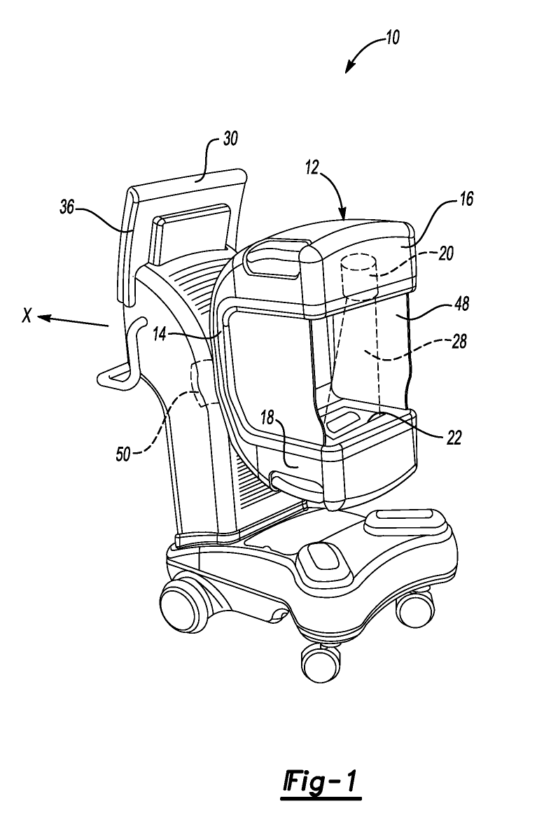

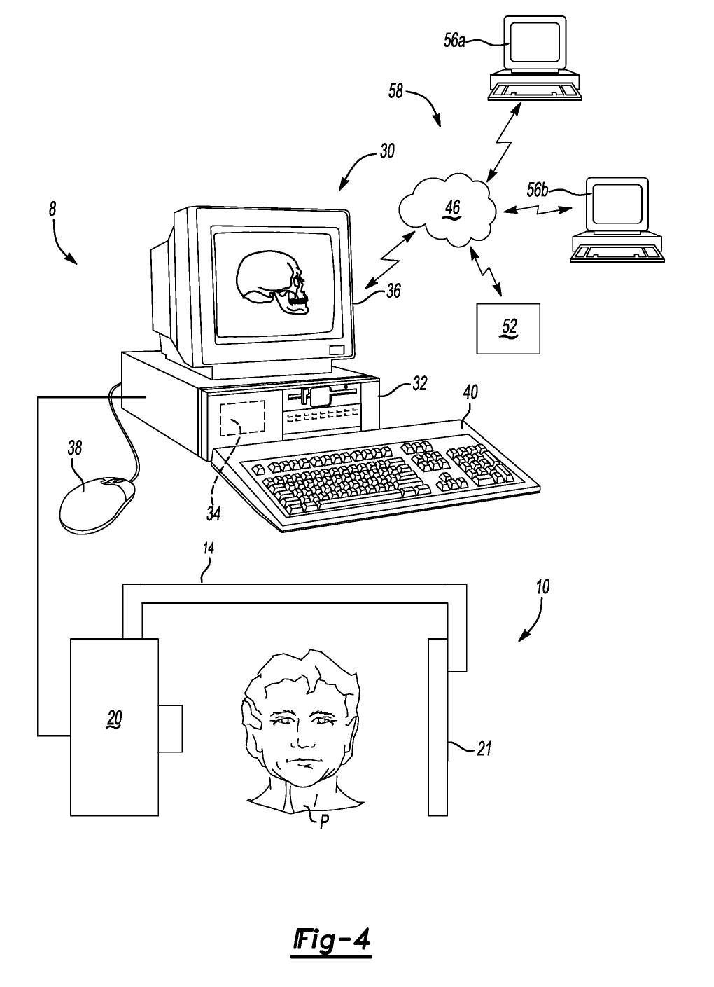

FIG. 1 illustrates a CT scanner 10 of a CT scanner system 8 (shown in FIG. 4) including a gantry 12 that supports and houses components of the CT scanner 10. Suitable CT scanners 10 are known. In one example, the gantry 12 includes a cross-bar section 14, and a first arm 16 and a second arm 18 each extend substantially perpendicularly from opposing ends of the cross-bar section 14 to form the c-shaped gantry 12. The first arm 16 houses an x-ray source 20 that generate x-rays 28. In one example, the x-ray source 20 is a cone-beam x-ray source. The second arm 18 houses a complementary flat-panel detector 22 spaced apart from the x-ray source 20. The x-rays 28 are directed toward the detector 22 which includes a converter (not shown) that converts the x-rays 28 from the x-ray source 20 to visible light and an array of photodetectors behind the converter to create an image. As the gantry 12 rotates about the patient P, the detector 22 takes a plurality of x-ray images at a plurality of rotational positions. Various configurations and types of x-ray sources 20 and detectors 22 can be utilized, and the invention is largely independent of the specific technology used for the CT scanner 10.

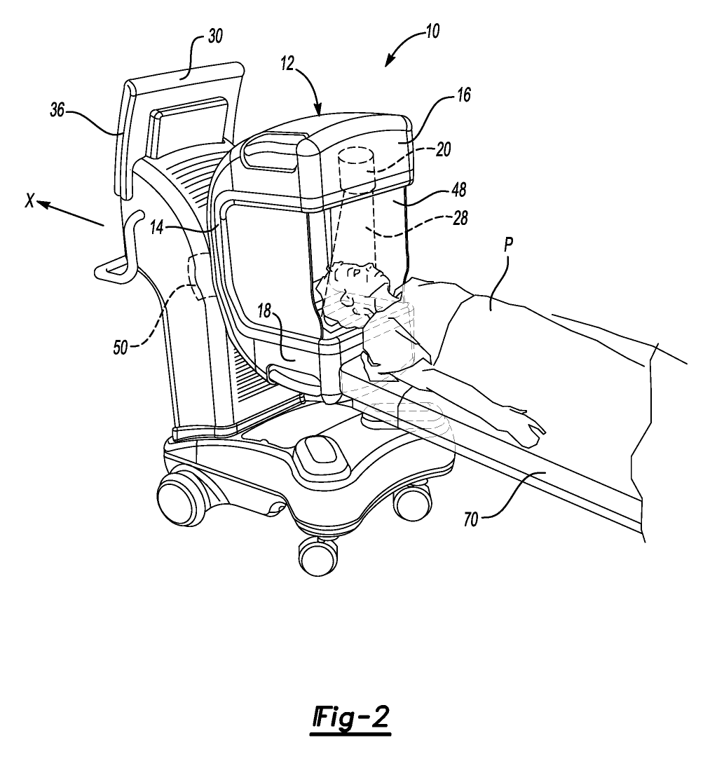

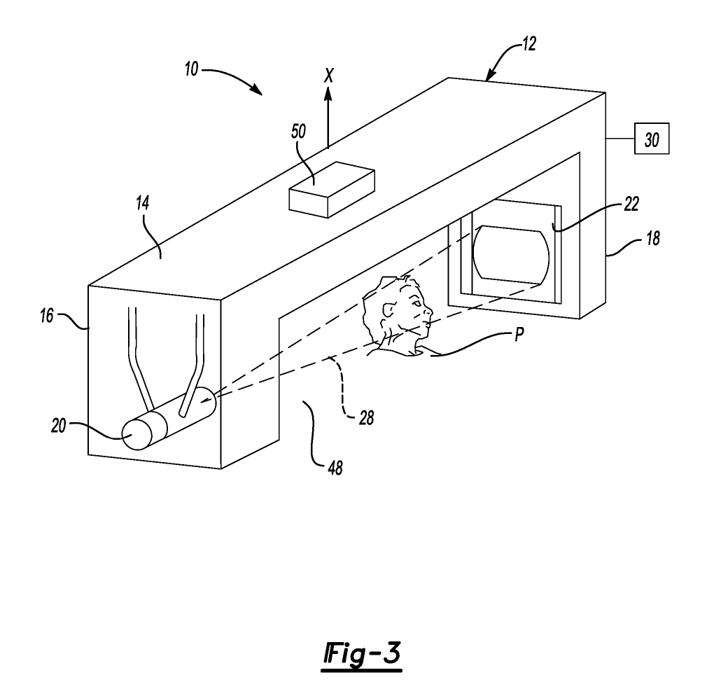

FIG. 2 illustrates the CT scanner 10 with a part of the patient P received in a space 48 between the first arm 16 and the second arm 18. A motor 50 rotates the gantry 12 about an axis of rotation X to obtain a plurality of x-ray images of the patient P at the plurality of rotational positions. The axis of rotation X is positioned between the x-ray source 20 and the detector 22. The gantry 12 can be rotated approximately slightly more than 360 degrees about the axis of rotation X. In one example, as shown in FIGS. 1 and 2, the axis of rotation X is substantially horizontal. In this example, the patient P is typically lying down on a table 70. Alternatively, as shown in FIG. 3, the axis of rotation X is substantially vertical. Typically, in this example, the patient P is sitting upright.

As shown schematically in FIG. 4, a CT scanner system 8 further includes a computer 30 having a microprocessor or CPU 32, a storage 34 (memory, hard drive, optical, and/or magnetic, etc. or other local storage), a display 36, a mouse 38, a keyboard 40 and other hardware and software for performing the functions described herein. The computer 30 powers and controls the x-ray source 20 and the motor 50. The plurality of x-ray images taken by the detector 22 are sent to the computer 30 and stored. The computer 30 generates a three-dimensional CT image from the plurality of x-ray images utilizing any known techniques and algorithms. The three-dimensional CT image is stored on the storage 34 of the computer 30 and can be displayed on the display 36 for viewing. An operator controls the CT scanning system 8 using the computer 30

The CT scanning system 8 includes an image exchange system 58. The computer 30 (or a separate computer) interfaces with an image exchange server 52 over the Internet or over another wide area network 46. The image exchange server 52 is accessible to other authorized users'computers 56a and 56b, including other CT scanning systems and other users, via the wide area network 46.

The image exchange server 52 generally provides three features: 1) an offsite backup of images; 2) complete project management for medical procedures involving the images; and 3) software available for trial and purchase for use on the CT scanning system 8 or other computers. Although the image exchange system 58 of the present invention is illustrated for use with a CT scanning system 8, it should be understood that other medical imaging devices, including but not limited to MRI and ultrasound devices, may employ the image exchange system 58 of the present invention.

The CT scanner 10 takes a plurality of x-ray images that are used to create the CT images. In operation, copies of the CT images stored on the computer 30 are periodically and automatically copied by software on the computer 30 to the image exchange server 52 and associated with a physician's account. The CT images on the image exchange server 52 are accessible only by the physician's account and those accounts authorized by the physician. This provides an automatic offsite backup of the CT images, while relieving the physicians of the time, effort and cost necessary to make periodic backups for offsite storage.

Once a CT image of the patient P is uploaded onto the image exchange server 52, the physician handling the patient P may, using the computer 30 or another computer, grant the authority to remotely view selected CT images through the image exchange server 52 to the patient, other physicians, or specialists. The physician does this by granting access to the selected CT images to the associated accounts of the other people. The image exchange server 52 includes a database of numerous accounts for all of the people who require access. The physician can access the database to find the people to whom access should be granted, or, especially in the case of a new patient, create a new account (user ID and password) to which access can be granted.

The physician also manages the project involving the selected CT image using the image exchange server 52, including 1) choosing outside contractors (other physicians, specialists, implant manufacturers, etc); 2) requesting tasks to be performed by the outside contractors relating to specific CT images; 3) monitoring responses (acceptance or rejection) from outside contractors to task requests; 4) monitoring the progress of the outside contractors on the requested tasks. The image exchange server 52 also coordinates among the outside contractors as necessary, for example, by notifying one contractor when a prerequisite work has been completed by another contractor. The image exchange server 52 may also optionally coordinate billing and payment among the physician, patient, insurance company and outside contractors for the services performed in connection with the CT images. One example project is described below.

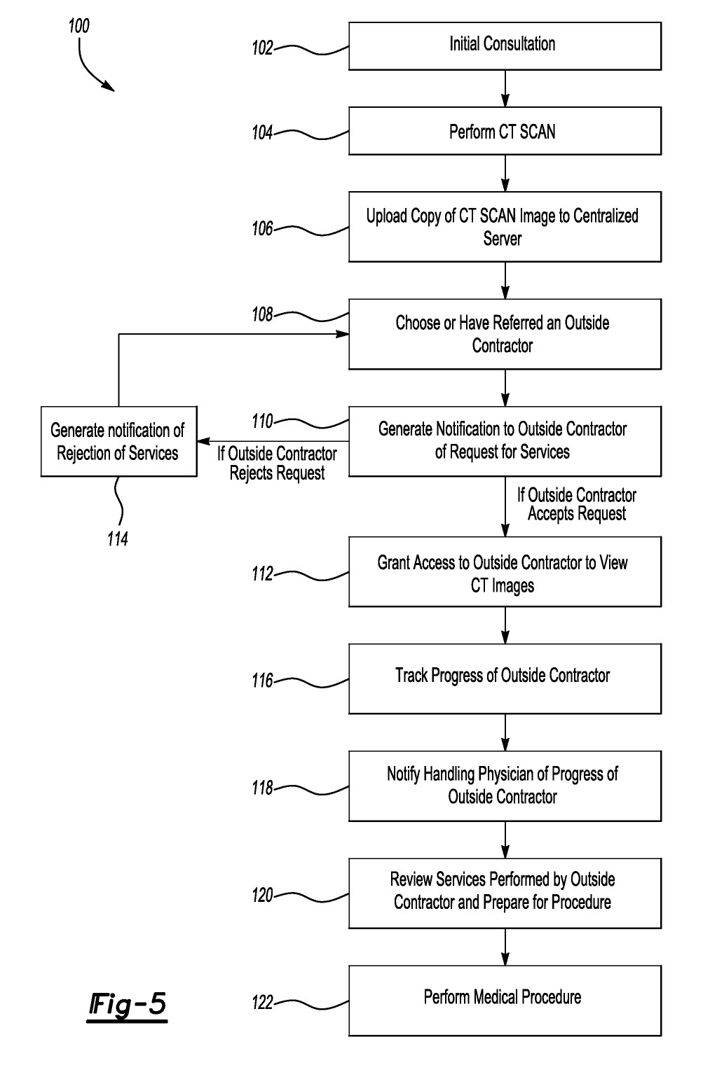

Referring to FIG. 5, a method 100 for storing, accessing and distributing CT images through the image exchange server 52 is illustrated. The following method 100 is illustrated in terms of a dental implant procedure. Although the example is illustrated in terms of a dental implant procedure, it should be understood that the method may be utilized in other situations including, but not limited to, procedures involving the temporal bones, skull base, or sinuses.

The method 100 begins at step 102 where a physician conducts an initial consultation with a patient P, and the physician determines that the surgical installation of a unique dental implant is necessary. Next, at step 104, the appropriate CT scans of the patient P are obtained, and a copy of the CT image obtained from the CT scan is uploaded onto the image exchange server 52 at step 106. At step 108, the physician handling the patient P (i.e., the handling physician) requests additional services to be performed by outside contractors (i.e., other doctors, surgeons, radiologists, specialists, etc.) based upon the needs of the patient as determined from the CT images.

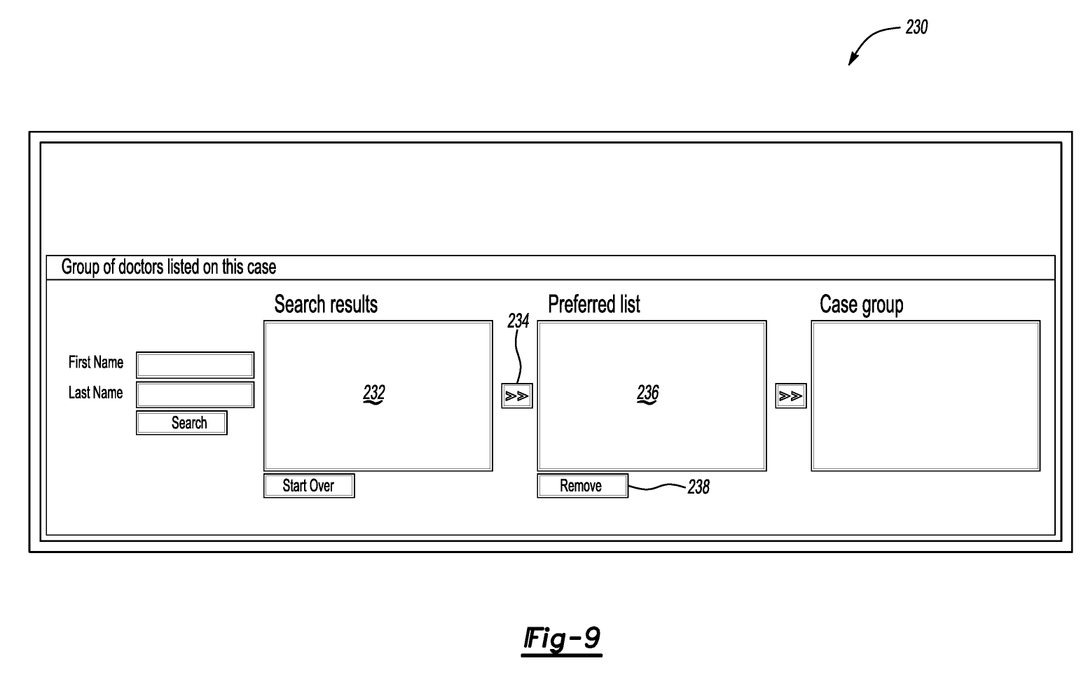

The handling physician may either request specific outside contractors to perform specific tasks (from a list of available outside contractors with accounts on image exchange server 52) or request referrals from the image exchange server 52. The handling physician may grant authorization to any person or organization with an account on image exchange server 52 to access selected CT images (see FIG. 9).

It is expected that physicians would usually be repeatedly initiating the same project for many different patients. In that case, the physician would store his preferences for outside contractors for each of the tasks of the project so that the physician could simply choose that project for a selected CT image, and the preferred outside contractors would automatically be assigned.

When referrals for an outside contractor in a selected category are requested by the handling physician, the image exchange server 52 displays a list of qualified outside contractors on the display 36 of the computer 30 to the handling physician. The handling physician and/or the patient P may then choose the outside contractor that best fit their needs. That is, the image exchange server 52 includes a complete referral network as a component of its project management capabilities, thereby further simplifying the procedure for both physicians and patients.

In step 110, the image exchange server 52 issues a notification to the outside contractors that access to the selected CT image(s) has been granted to them and that specific work on the project is requested of them. The notification may be done through the outside contractors'accounts on the image exchange server 52 and/or via outside email. The outside contractor access the image exchange server 52 using computers 56a and 56b over the wide area network 46. The outside contractor may either accept or reject the request, and the physician is notified of the acceptance or rejection.

Upon acceptance of the request to perform services, the image exchange server 52 grants the outside contractor access to the CT images of the patient P at step 112. The outside contractors may then add comments to the CT images, mark up the CT images, design implants based upon the CT images, locate implants within the CT images or perform any other required services on or with the CT images. The handling physician can select several outside contractors and select the specific images to which each outside contractor has access. For example, the handling physician restricts access such that each outside contractor only has access to specific images.

In the event the outside contractor rejects the handling physician's request to perform services, the image exchange server 52 issues a notification indicating such rejection to the handling physician at step 114. The method 100 then returns to step 108 where a second outside contractor is chosen to perform the required services. Alternatively, the image exchange server 52 may automatically contact alternative outside contractors approved by the physician upon the rejection by the first outside contractor.

At step 116, the image exchange server 52 tracks the progress of the outside contractors as the requested services are performed. The image exchange server 52 notifies the handling physician of the progress of the outside contractor at step 118. For example, the image exchange server 52 may send a series of email progress reports to the handling physician. Alternatively, a listing of all of the physician's open projects may be displayed in a single list along with a summary of the progress of each project. Additionally, since some tasks are typically performed sequentially by several outside contractors, the image exchange server 52 may notify one outside contractor of another outside contractor's progress to date or notify the outside contractor when the work of a previous outside contractor is completed. Because the outside contractors may be working on many projects for many different physicians, a listing of their active projects may be displayed to them, along with a status indicator, priority indicator, deadline, etc.

For example, in the dental implant field, the CT images may need to be reviewed by a second physician, a radiologist, a drill template manufacturer and an implant manufacturer. First, the patient may request a second physician to review the CT image for a second opinion. Also, a radiologist may be required to review the CT image to screen for potential issues, such as tumors. These tasks can be assigned and performed simultaneously. Once these parties have completed their review of the CT images and made all necessary annotations (or simultaneously), the image exchange server 52 sends a notification to the implant manufacturer that the CT image is available to access. The implant manufacturer accesses the CT image and designs an appropriately sized dental implant for a patient P. The implant design is added to the CT image (as a separate layer, or in an otherwise non-destructive manner). Once the implant manufacture has completed the implant design, a notification is sent to the handling physician requesting approval and/or comments on the implant design. After approval, or automatically, notification is given to the drill template manufacturer, who designs a template based upon the CT image and based upon the implant design, which has been added to the CT image. By tracking all markups and the progress of every party involved in completion of the medical procedure, the image exchange server 52 acts as a project manager and effectively streamlines the entire surgical procedure.

At step 120, the handling physician reviews the services performed by all outside contractors and makes any necessary preparations for performance of the required medical procedure. The implant and template are shipped to the physician. The method 100 ends at step 122 where the medical procedure is completed.

One additional capability of the image exchange server 52 is the ability to coordinate payments. For example, as stated above, the dental implant field often requires the services of a second physician, a radiologist, a drill template manufacturer, and an implant manufacturer. All of these outside contractors require a fee for the services they provide. The image exchange server 52 may be programmed with the necessary software to control the money transactions involved in paying for these outside contractor's services. One of ordinary skill in the art with the benefit of the teachings herein will be able to implement the appropriate instructions to facilitate the execution of money transactions.

In one example, the patient P logs onto the image exchange server 52 and provides credit card information. The image exchange server 52 then automatically deducts funds from the credit card of the patient P and pays the appropriate outside contractor via an electronic money transfer once authorization is given that the services were adequately completed. In another example, insurance information is programmed into the image exchange server 52 such that an automatic bill is produced and sent by the image exchange server 52 to the relevant insurance agency once the outside contractor's services are completed.

In addition to storage capabilities, the image exchange server 52 also includes numerous types of software that can be downloaded to the computer 30 for trial or purchase, including implant planning software, commenting software or any other CT scan related software. Software purchased from the image exchange server 52 is billed to the account associated with the computer 30, 56a and 56b that downloaded it.

Additionally, the network can prioritize how the images are transferred from the image exchange server 52 to the secondary contractor's computers 56a and 56b. Numerous images for each patient can be stored on the image exchange server 52, and therefore it can be time consuming to transfer the images to the secondary user's computers 56a and 56b. For example, when the secondary contractor views the images, he can select an image of increased priority. The image exchange server 52 will first transfer this image to the secondary contractor's computer 56a and 56b, allowing the outside contractor to begin reviewing and annotating the image. As the outside contractor continues to review the image, the remaining images can be transferred to the outside contractor's computer 56a and 56b. This speeds up the transfer of the higher priority images to the outside contractor's computer 56a and 56b so he can begin work immediately.

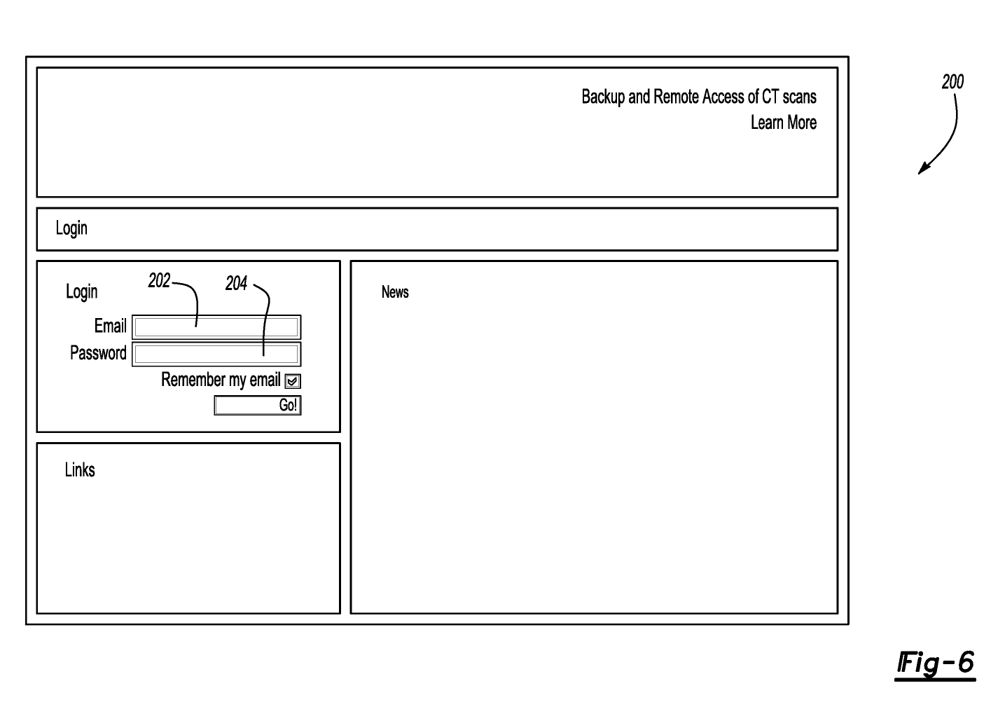

FIGS. 6 to 9 illustrate screen captures shown on the display 36 of the computer 30 (or any computer) when using the software and accessing the imaging exchange network. For example, as shown in FIG. 6, the user first sees a login page 200. On the login page 200, the user inputs an email address in a field 202 and a password in a field 204 to access the imaging exchange network.

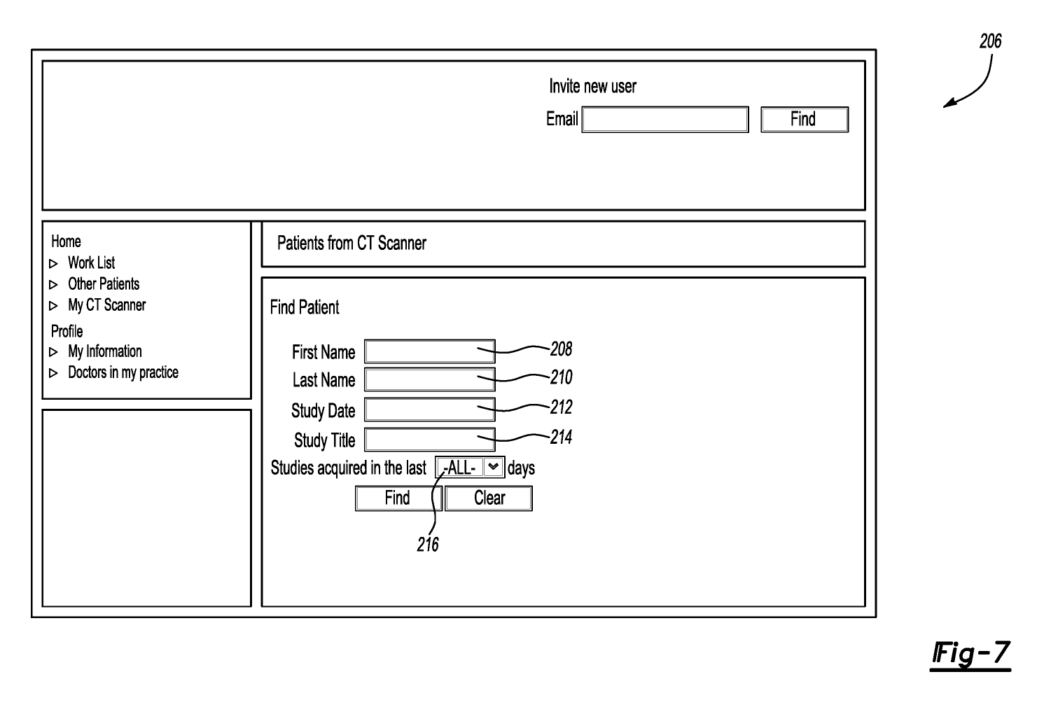

As shown in FIG. 7, the user can then access a patient search page 206 where the user can input information to search for a patient's P records. A patient's P records can be accessed by inputting a first name in a field 208, a last name in a field 210, a study date in a field 212 or a study title in a field 214. The user can also select all studies acquired in a given time period using a drop-down menu 216.

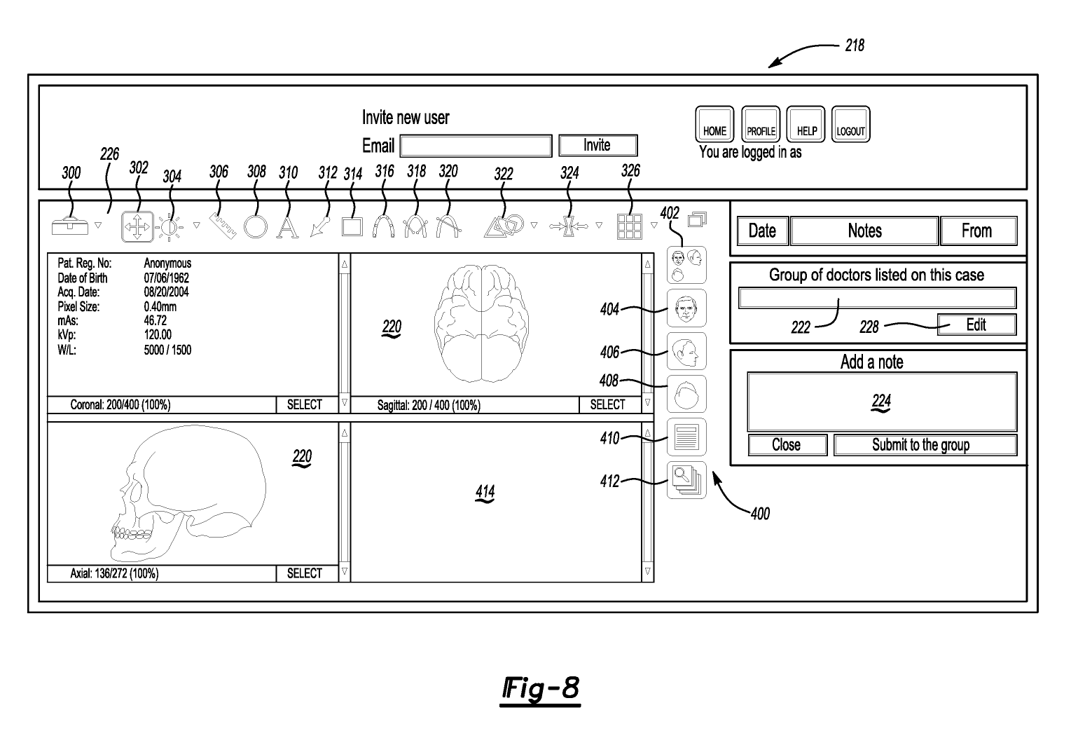

As shown in FIG. 8, once the records of the patient P are identified, the user is then taken to an image page 218 that displays the images 220 of the patient P. The group of physicians, outside contractors and authorized users that are authorized to access the images 220 is shown in a field 222. Notes about the images 220 can be made by the outside contractor in a section 224. The images 220 can also be marked-up, reviewed and noted by the outside contractor using a tools section 226, allowing the outside contractor to perform the requested tasks.

For example, the tools section 226 can include several icons that the outside contractor can use to perform the requested tasks. Example icons include a viewing tools icon 300 to reset the screen, a zoom/unzoom/drag icon 302 to enlarge, reduce or move a selected image, a contrast icon 304 to adjust the contrast of the selected image and a measurement icon 306 to measure a selected portion of the selected image. The tools section 226 can also include an icon 308 that is clicked to determine a mean/standard deviation density in an area of interest (circled area), a label icon 310 to label the selected image, a label with arrow icon 312 to label the selected image, and an icon 314 to determine the mean/standard deviation density in an area of interest (squared area). The tools section 226 can also include an icon 316 for panoramic tracing, an icon 318 for temporomandibular tracking, and an icon 320 for single plane tracing. The tools section 226 can further include a label list icon 322 that is clicked to provide a list of labels that can be used by the user to annotate the selected image, a data compression icon 324 that can be clicked to compress the data and a column display icon 326 to show the data in a column.

The specific tools/icons displayed in the tools section 226 on the image page 218 can be customized based on the user. For example, if a user in the dental field is using the software, the software is programmed such that the tools needed specifically for dental images are active. Any non-essential tools can be darkened and inactivated or removed.

FIG. 8 also shows a viewing section 400 of the image page 218. The viewing section 400 includes icons that can be clicked to select a desired view of the image. The viewing section 400 includes a quadrant icon 102 to view several views of the image (default image), a frontal icon 404 to view a front view of the image, a sagittal icon 406 to view a side view of the image, an axial icon 408 to view a top view of the image, a CT data parameter icon 410 to view the parameters of the CT data, and a selected sections icon 412. The selected sections icon 412 displays sections pre- programmed by a user.

The quadrant 414 can display a three-dimensional rendering of the image. Tools can be provided to change the view of the three-dimensional image and perform any requested tasks on the three-dimensional image.

Although numerous icons and tasks are illustrated and described, it is to be understood that any combination of the icons/tasks can be used or other icons can be added or deleted based on the desired task.

To change the list of people with access to the patient's P records, the physician clicks the edit button 228, taking the physician to the outside contractor editing page 230 shown in FIG. 9. The physician can add outside contractors to the authorization list by inputting or selecting the names of the outside contractors in a field 232. An arrow 234 can be clicked to add the outside contractors to the “preferred list” shown in a field 236. An individual on the “preferred list” can be removed by highlighting the outside contractor's name and clicking the remove button 238.

The foregoing description shall be interpreted as illustrative and not in any limiting sense. A worker of ordinary skill in the art would recognize that certain modifications would come within the scope of this invention. For that reason, the following claim should be studied to determine the true scope and content of this invention.

What is claimed is:

1. A method for managing an image of a patient, the method comprising the steps of:
generating an image of a patient;
storing the image on a remote centralized server;
authorizing at least one user to remotely access the image; and
remotely accessing the image.

2. The method as recited in claim 1 further including the step of annotating the image.

3. The method as recited in claim 2 further including the step of selecting the at least one user, wherein the at least one user performs the steps of remotely accessing the image and annotating the image.

4. The method as recited in claim 2 wherein the at least one user comprises a first outside contractor and a second outside contractor, the method further including the step of notifying the second outside contractor when the first outside contractor completes the step of annotating the image.

5. The method as recited in claim 2 further including the step of reviewing annotations provided by the at least one user.

6. The method as recited in claim 5 further including the step of determining a procedure to perform on the patient based on the step of reviewing the annotations.

7. The method as recited in claim 1 wherein the step of generating the image of the patient includes taking a plurality of x-ray images to create a CT image.

8. The method as recited in claim 1 further including the steps of storing the image on a computer and uploading the image from the computer to the remote centralized server.

9. The method as recited in claim 1 further including the steps of storing the image on a first computer and uploading the image from the first computer to the remote centralized server before the step of storing the image on the remote centralized server, wherein the step of remotely accessing the image is performed on a second computer by the at least one user.

10. The method as recited in claim 9 wherein an originating physician uses the first computer and the at least one user is an outside contractor that uses the second computer.

11. The method as recited in claim 1 further including the step of requesting the at least one user to review the image.

12. The method as recited in claim 11 further including the step of accepting a request to review the image.

13. The method as recited in claim 11 further including the steps of rejecting a request to review the image and then choosing another at least one user to review the image.

14. The method as recited in claim 1 further including the step of monitoring work performed by the at least one user.

15. The method as recited in claim 1 further including the step of coordinating payments to the at least one user.

16. The method as recited in claim 1 further including the step of downloading software from the remote centralized server to a computer.

17. The method as recited in claim 1 wherein the image comprises a primary image and a plurality of secondary images, the method further including the steps of selecting the primary image, transferring the primary image to a computer of the at least one user, and reviewing the primary image while the plurality of secondary images transfer to the computer of the at least one user.

18. The method as recited in claim 1 wherein the image comprises a first image and a second image and the at least one user comprises a first user and a second user, wherein the step of authorizing the at least one user includes authorizing the first user to remotely access at least the first image and authorizing the second user to remotely access at least the second image.

19. A method for managing a CT image of a patient, the method comprising the steps of:
taking a plurality of x-ray images to create a CT image of a patient;
uploading the CT image to a remote centralized server;
storing the CT image on the remote centralized server;
authorizing at least one outside contractor to remotely access the CT image;
remotely accessing the CT image;
annotating the CT image; and
monitoring work performed by the at least one outside contractor.

20. The method as recited in claim 19 further including the step of selecting the at least one outside contractor, wherein the at least one outside contractor performs the steps of remotely accessing the CT image and annotating the CT image.

21. The method as recited in claim 19 further including the step of requesting the at least one outside contractor to review the image.

22. The method as recited in claim 21 further including the step of accepting a request to review the image.

23. The method as recited in claim 21 further including the steps of rejecting a request to review the image and then choosing another at least one outside contractor to review the image.

24. The method as recited in claim 19 further including the step of reviewing annotations provided by the at least one outside contractor.

25. The method as recited in claim 19 further including the steps of storing the image on a first computer, and the step of uploading the image to the remote centralized server includes uploading the image from the first computer to the remote centralized server, wherein the step of remotely accessing the image is performed on a second computer by the at least one outside contractor.

26. The method as recited in claim 25 wherein an originating physician uses the first computer and the at least outside contractor uses the second computer.

27. The method as recited in claim 19 further including the step of coordinating payments to the at least one outside contractor.

28. The method as recited in claim 19 further including the step of downloading software from the remote centralized server to a computer.

29. A surgical imaging system comprising:
an image device to take an image of a patient;
a first computer to store the image;
a remote centralized server, wherein the first computer interfaces with the remote centralized server and uploads the image to the remote centralized server; and
a second computer, wherein the second computer interfaces with the remote centralized server to remotely access the image, a first user authorizes a second user to remotely access the image, and the second user annotates the image on the second computer.

30. The surgical imaging system as recited in claim 29 wherein the first computer and the second computer interface with the remote centralized server over a wide area network.

31. The surgical imaging system as recited in claim 29 wherein the image device is a CT scanner and the image is a CT image.

32. The surgical imaging system as recited in claim 29 wherein the first user uses the first computer to monitor work performed by the second user on the second computer.

33. The surgical imaging system as recited in claim 29 wherein the first user uses the first computer to review annotations provided by the second user on the second computer.
